(19) United States
(12) Patent Application Publication
(10) Pub. No.: US 2022/0133372 A1
(43) Pub. Date: May 5, 2022

(54) EPIPHYSEAL PLATE CLAMPING DEVICE

(71) Applicant: Chang Gung Memorial Hospital, Linkou, Taoyuan City (TW)

(72) Inventors: Chia-Hsieh CHANG, Taoyuan City (TW); Chih-Chung HU, New Taipei City (TW)

(21) Appl. No.: 17/497,644

(22) Filed: Oct. 8, 2021

(30) Foreign Application Priority Data
Nov. 2, 2020 (TW) 109138121

(51) Int. Cl. A61B 17/80

(52) U.S. Cl. CPC A61B 17/8061

(57) ABSTRACT

An epiphyseal plate clamping device, comprising: a pair of clamping pieces each having at least two through holes; and at least two screw assemblies to correspond to the at least two through holes of the pair of clamping pieces; where, the pair of clamping pieces are used to be placed upon two sides of an epiphyseal plate zone of a longer leg of an LLD patient, and the at least two screw assemblies are used to penetrate through a femur or tibia of the longer leg along at least two paths guided by the at least two through holes of the pair of clamping pieces to clamp an epiphyseal plate within the epiphyseal plate zone, thereby retarding a growth of new bone from the epiphyseal plate.

### BACKGROUND OF THE INVENTION

### Field of the Invention

The present invention relates to an epiphyseal plate clamping device, in particular to a clamping device that can symmetrically slow down the growth of new bone from the epiphyseal plate.

### Description of the Related Art

Leg length discrepancy (LLD) is a pathological condition from the difference of actual bone lengths of two legs.

Since the difference in the length of the legs of LLD children is likely to increase with age, it is advisable to give LLD children appropriate treatment as soon as possible, and the general early treatment is epiphysiodesis at the longer leg.

Epiphysiodesis is a surgical treatment to stop or slow down bone growth in children. There are two types of epiphysiodesis: permanent and temporary. Permanent epiphysiodesis refers to the ablation of the epiphyseal plate to stop bone growth. A satisfactory result depends on an accurate estimation of the timing of permanent epiphysiodesis because the procedure is irreversible. Temporary epiphysiodesis slows down bone growth through implants but preserves the epiphyseal plate. The growth suppression can be stopped by removing the implants once the treatment goal has been achieved.

Several implants have been developed to suppress bone growth temporarily, including the use of staples, tension band plates, and percutaneous transphyseal screws. Implant-related complications such as loosening, malposition, broken screws, and angular deformity have been reported. After temporary epiphysiodesis, the angular deformity was reported to occur in as many as 50% of cases using staples and 20% using screws. Asymmetric suppression of the epiphyseal plate between the medial and lateral implants is responsible of angular deformity. Therefore, there is a need for a novel epiphyseal plate clamping device in the art.

### SUMMARY OF THE INVENTION

One objective of the present invention is to provide an epiphyseal plate clamping device, which can be conveniently operated by a surgeon to install a longitudinal clamping structure along the direction of epiphyseal plate growth at the longer leg, thereby solving the leg length discrepancy problem of the patient.

Another objective of the present invention is to provide an extensible epiphyseal plate clamping device, which can install a longitudinal tethering effect for epiphyseal plate growth, thereby avoiding tissue damage in the epiphyseal plate from too much compression and avoiding implant loosening from continuous epiphyseal plate growth.

Another objective of the present invention is to provide a design that couples a medial device, which refers to a device installed at the inner side of a femur or tibia, with a lateral device, which refers to a device installed at the outer side of the femur or tibia, to produce symmetrical tethering of epiphyseal plate growth, thereby avoiding bone deformity from asymmetric suppression of the epiphyseal plate.

Still another objective of the present invention is to provide a guiding system to accurately install the device with minimum surgical injury.

To attain the aforementioned objectives, an epiphyseal plate clamping device is proposed, which includes:
a pair of clamping pieces each having at least two through holes; and
at least two screw assemblies to correspond to the at least two through holes of the pair of clamping pieces;
where, the pair of clamping pieces are used to be placed upon two sides of an epiphyseal plate zone of a longer leg of an LLD patient, and the at least two screw assemblies are used to penetrate through a femur or tibia of the longer leg along at least two paths guided by the at least two through holes of the pair of clamping pieces to clamp an epiphyseal plate, thereby retarding a growth of new bone from the epiphyseal plate.

In one embodiment, at least one aforementioned screw assembly has an internally threaded sleeve and an elongated screw, the elongated screw having a length greater than a width of the longer leg at the epiphyseal plate, and when in operation, the elongated screw penetrates through the femur or tibia via a guiding path provided by a guiding pin, which is previously inserted through the femur or tibia and two corresponding through holes of the pair of clamping pieces at opposite sides of the femur or tibia, to be engaged with the internally threaded sleeve.

In one embodiment, at least one aforementioned screw assembly has a guiding sleeve disposed in one of the through holes of one aforementioned clamping piece for the elongated screw to pass through.

In one embodiment, at least one aforementioned screw assembly has a pair of screws, and when in operation, the pair of screws are screwed into the femur or tibia through two aforementioned through holes of the pair of clamping pieces that are disposed at opposite sides of the femur or tibia.

For possible embodiments, the elongated screw and the pair of screws may be made of stainless steel or titanium alloy.

For possible embodiments, the guiding sleeve can be permanently or detachably combined with the through hole.

For possible embodiments, the internally threaded sleeve can be permanently or detachably combined with the through hole.

In one embodiment, the pair of clamping pieces is extensible.

In one embodiment, the pair of clamping pieces each has a stretchable frame body.

For possible embodiments, the stretchable frame body can be made of stainless steel or titanium alloy.

To make it easier for our examiner to understand the objective of the invention, its structure, innovative features, and performance, we use preferred embodiments together with the accompanying drawings for the detailed description of the invention.

### BRIEF DESCRIPTION OF THE DRAWINGS

FIG. 1 illustrates a schematic diagram of an embodiment of the epiphyseal plate clamping device of the present invention.

FIG. 2 illustrates a schematic diagram of the parts of the epiphyseal plate clamping device of FIG. 1 that are combined with a femur or tibia during a surgical operation.

FIG. 3 illustrates a schematic diagram of the parts of the epiphyseal plate clamping device of FIG. 1 that are combined with a femur or tibia after a surgical operation.

FIG. 4 illustrates a schematic diagram of another embodiment of the epiphyseal plate clamping device of the present invention.

FIG. 5 illustrates a schematic diagram of the parts of the epiphyseal plate clamping device of FIG. 4 that are combined with a femur or tibia during a surgical operation.

FIG. 6 illustrates a schematic diagram of the parts of the epiphyseal plate clamping device of FIG. 5 that are combined with a femur or tibia after a surgical operation.

FIG. 7 illustrates a schematic diagram of another embodiment of the epiphyseal plate clamping device of the present invention.

FIG. 8a illustrates a schematic diagram of a stretchable clamping piece utilized by the epiphyseal plate clamping device of the present invention.

FIG. 8b illustrates a scenario where the stretchable clamping piece shown in FIG. 8a is initially attached with an epiphyseal plate zone.

FIG. 8c illustrates a scenario where the stretchable clamping piece shown in FIG. 8b is stretched longer after the epiphyseal plate zone has grown new bone.

### DETAILED DESCRIPTION OF THE PREFERRED EMBODIMENTS

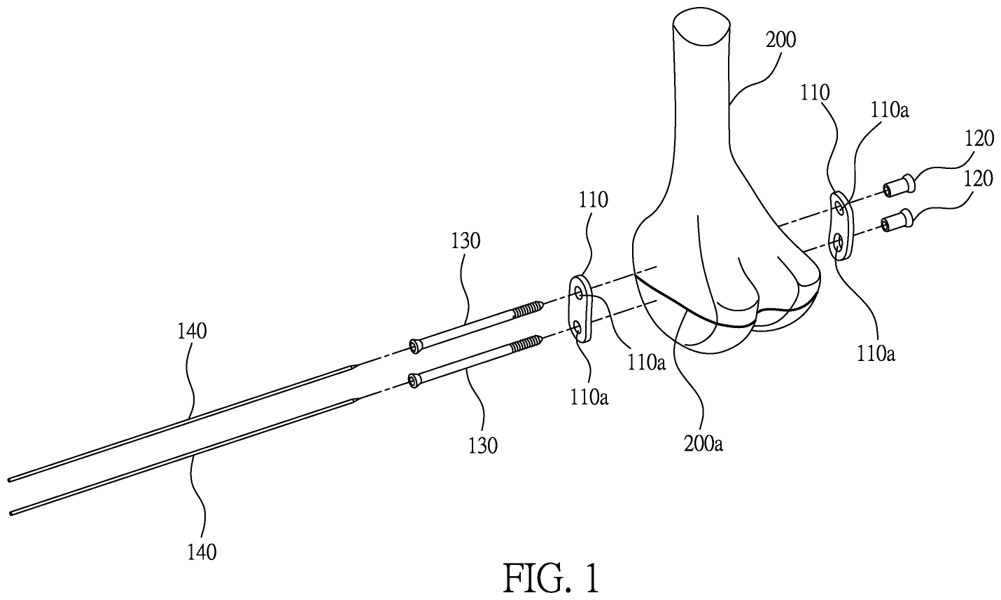

Please refer to FIG. 1, which illustrates a schematic diagram of an embodiment of the epiphyseal plate clamping device of the present invention. As shown in FIG. 1, the epiphyseal plate clamping device has a pair of clamping pieces 110, a pair of internally threaded sleeves 120 and a pair of elongated screws 130, where the pair of internally threaded sleeves 120 serves as a medial device and the pair of elongated screws 130 serves as a lateral device.

Each clamping piece 110 has two through holes 110a.

The pair of internally threaded sleeves 120 and the pair of elongated screws 130 form two screw assemblies to correspond to the two through holes 110a of the clamping pieces 110.

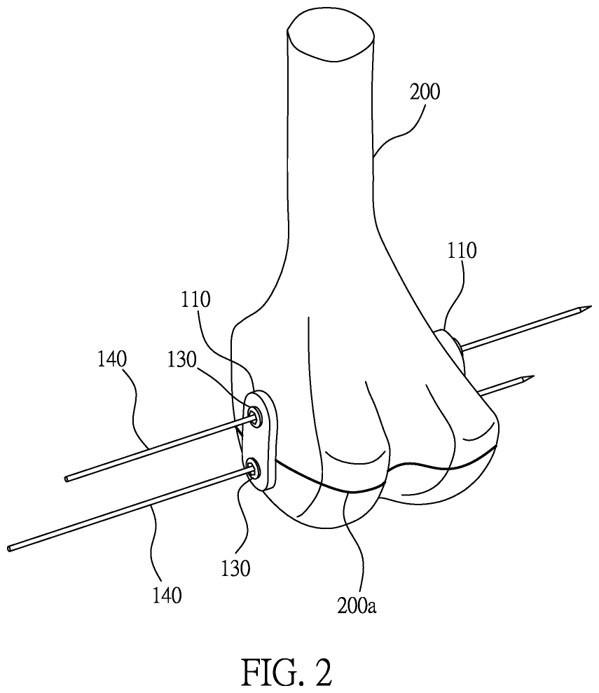

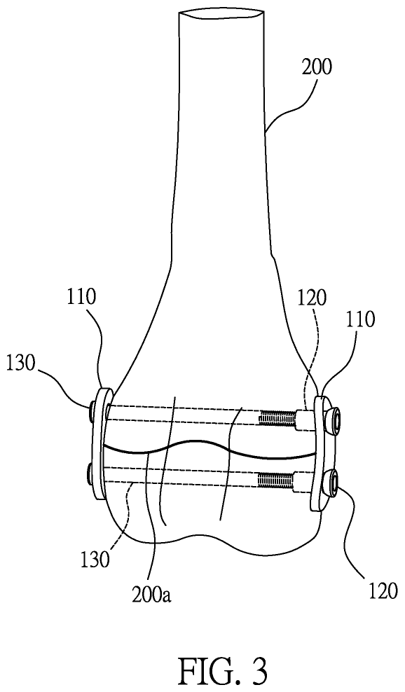

When in operation, please refer to FIG. 2, the pair of clamping pieces 110 are tightly attached to both sides of an epiphyseal plate zone 200a of a femur or tibia 200 of a long leg (relative to the other short leg), and the two screw assemblies are used to penetrate into two locations above and below the epiphyseal plate zone 200a of the femur or tibia 200 respectively via two through holes 110a of the pair of clamping pieces 110, that is, each of the screw assemblies has an internally threaded sleeve 120 and an elongated screw 130, the length of the elongated screw 130 is greater than the width of the femur or tibia 200 at the epiphyseal plate zone 200a, and the elongated screw 130 is used to penetrate through the femur or tibia 200 via a guiding path provided by a guiding pin 140, which is previously inserted through the femur or tibia 200 and two corresponding through holes 110a of the pair of clamping pieces 110 at opposite sides of the femur or tibia 200, to be engaged with the internally threaded sleeve 120. To be more specific, the guiding pin 140 is used to guide a drill to create a bone tunnel in the femur or tibia 200 for the elongated screw 130 to be inserted into, and the guiding pin 140 is withdrawn after the bone tunnel is completed. Thereafter, an elongated screw 130 can pass through a through hole 110a of a clamping piece 110 from one side of the bone tunnel to enter the bone tunnel, then pass through a through hole 110a of another clamping piece 110 on the other side of the bone tunnel, and eventually be engaged with an internally threaded sleeve 120. The scenario is illustrated in FIG. 3.

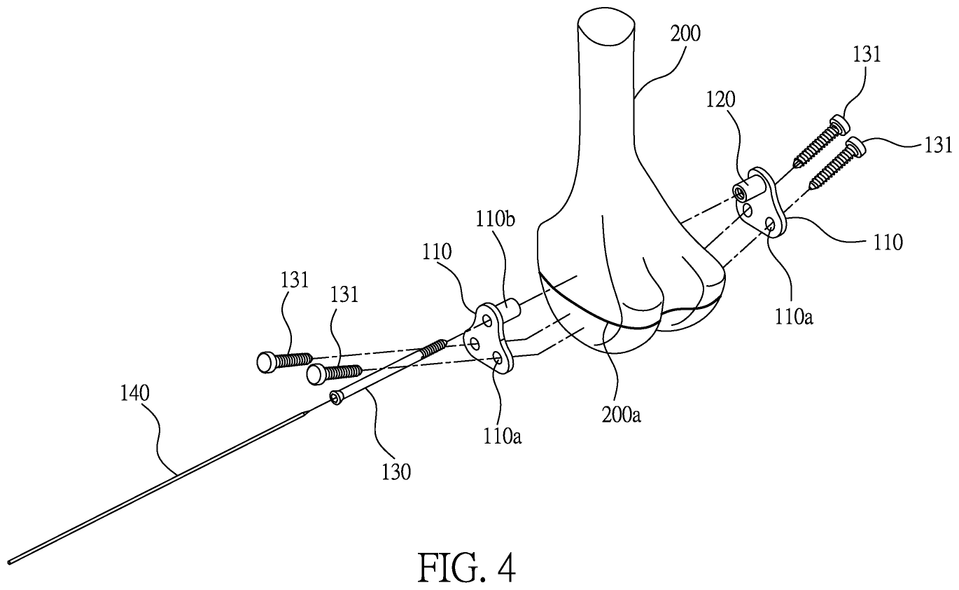

Please refer to FIG. 4, which illustrates a schematic diagram of another embodiment of the epiphyseal plate clamping device of the present invention. As shown in FIG. 4, the epiphyseal plate clamping device has a pair of clamping pieces 110, an internally threaded sleeve 120, an elongated screw 130 and two pairs of hollow screws 131.

Each clamping piece 110 has three through holes 110a, and one clamping piece 110 is provided with a guiding sleeve 110b in one through hole 110a thereof.

The internally threaded sleeve 120 and the elongated screw 130 form a first screw assembly to correspond to two respective through holes 110a of the pair of clamping pieces 110, where one of the two respective through holes 110a is provided with a guiding sleeve 110b.

Two pairs of hollow screws 131 form two second screw assemblies, and are used to screw into the bone 200 through two pairs of opposed through holes 110a of the pair of clamping pieces 110.

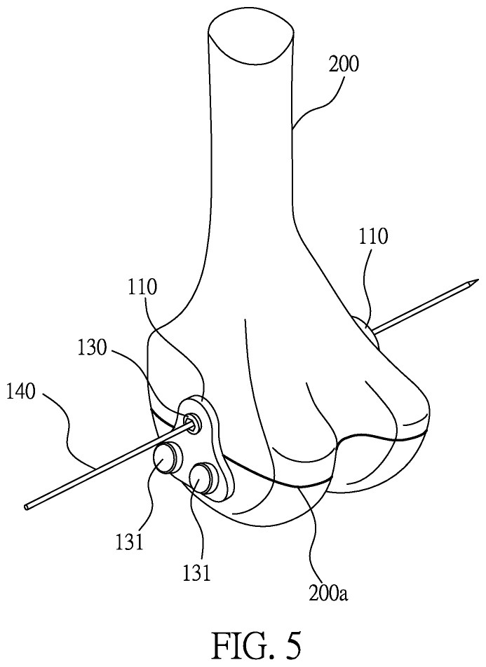

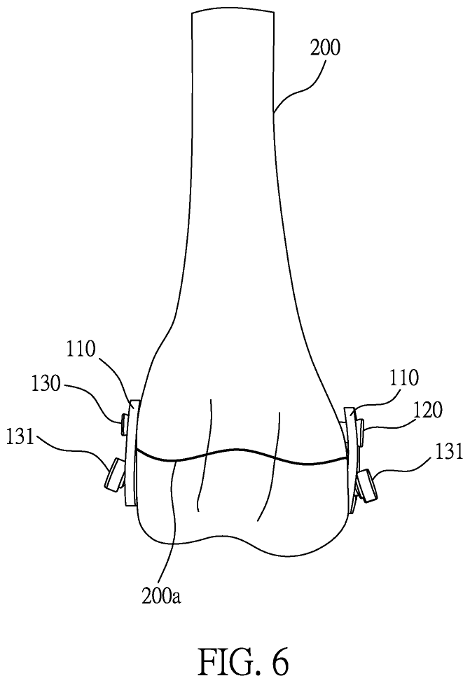

When in operation, please refer to FIG. 5, the pair of clamping pieces 110 are tightly attached to both sides of the epiphyseal plate zone 200a of the femur or tibia 200, and the first screw assembly and the two second screw assembly are used to pass three through holes 110a of the pair of clamping pieces 110 to penetrate into the femur or tibia 200 to clamp the epiphyseal plate zone 200a, where, the first screw assembly has an internally threaded sleeve 120, a guiding sleeve 110b and an elongated screw 130, the length of the elongated screw 130 is greater than the width of the femur or tibia 200 at the epiphyseal plate zone 200a, and the elongated screw 130 is used to penetrate through the femur or tibia 200 via a guiding path provided by a guiding pin 140, which is previously inserted through the femur or tibia 200 and two corresponding through holes 110a of the pair of clamping pieces 110 at opposite sides of the femur or tibia 200, to be engaged with the internally threaded sleeve 120; and two second screw assemblies formed by two pairs of hollow screws 131 are used to screw into the femur or tibia 200 through two pairs of through holes 110a of the pair of clamping pieces 100. Please refer to FIG. 6, which illustrates a schematic diagram of the screw assemblies engaged with the femur or tibia 200 after the operation is completed.

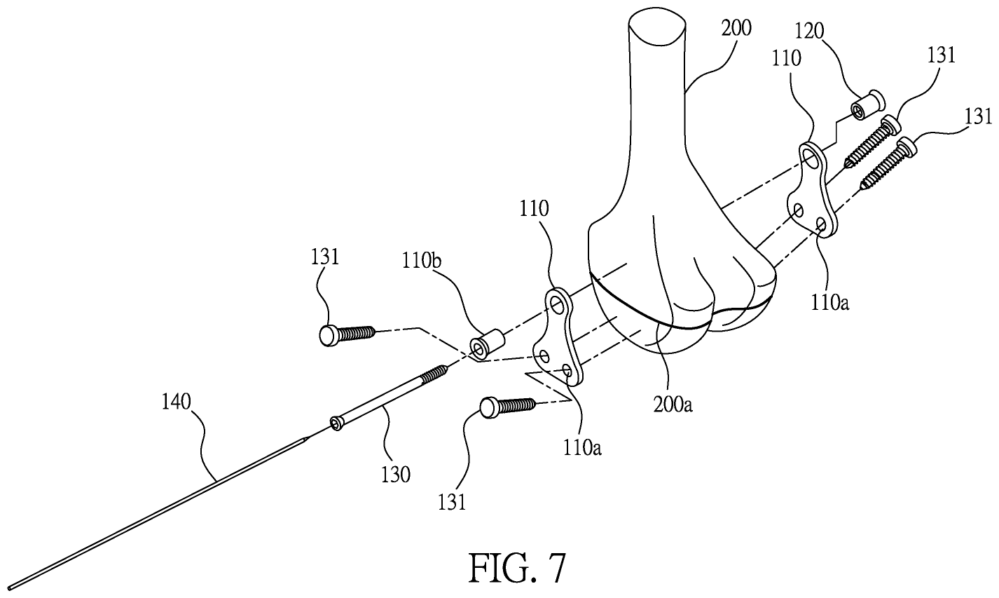

In addition, both the guiding sleeve 110b and the internally threaded sleeve 120 can be permanently or detachably combined with the through hole 110a. Please refer to FIG. 7, which illustrates a schematic diagram of still another embodiment of the epiphyseal plate clamping device of the present invention. As shown in FIG. 7, the guiding sleeve 110b and the internally threaded sleeve 120 of the epiphyseal plate clamping device are detachably combined with the through hole 110a.

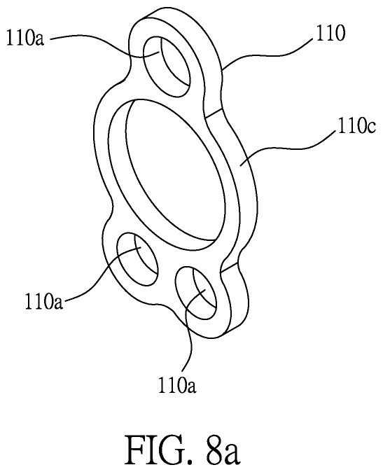

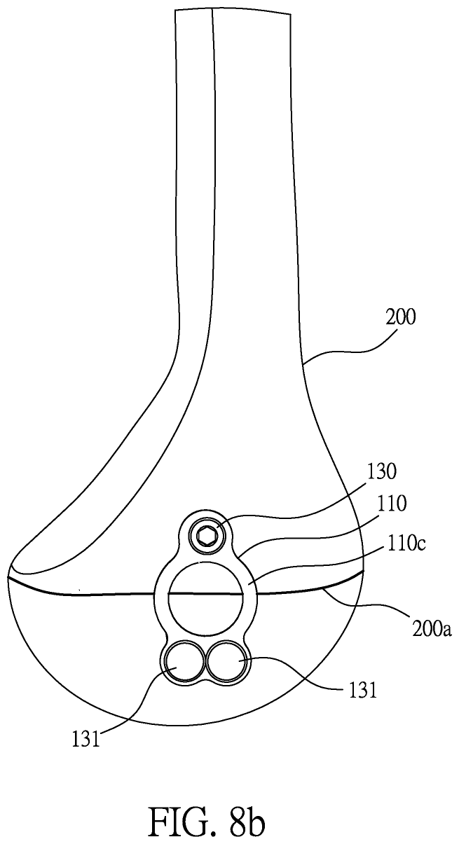

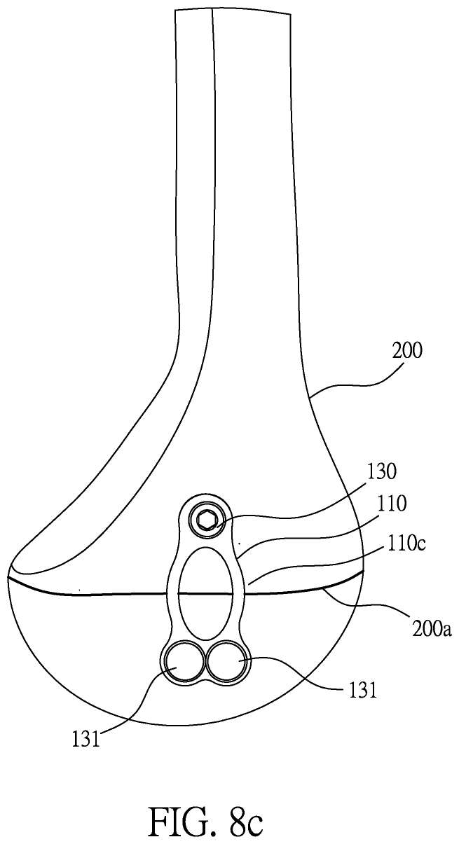

In addition, apart from providing a limiting effect on the epiphyseal plate growth, the clamping piece 110 can be stretchable to permit a limited epiphyseal plate growth. Please refer to FIG. 8a, which illustrates a schematic diagram of a stretchable clamping piece utilized by the epiphyseal plate clamping device of the present invention. As shown in FIG. 8a, the clamping piece 110 has a stretchable frame body 110c, which can be made of stainless steel or titanium alloy and can have different sizes for different ages of patients. When in use, the form of the stretchable frame body 110c of the clamping piece 110 is initially unchanged as illustrated in FIG.

8b, and the stretchable frame body 110c is stretched longer after a specific period of time as illustrated in FIG. 8c.

In summary, the description above has clearly explained the structure and principle of the epiphyseal plate clamping device of the present invention, and thanks to the novel designs thereof, the present invention possesses the following advantages:

(1) The epiphyseal plate clamping device of the present invention can be conveniently operated by a surgeon to install a longitudinal clamping structure along the direction of epiphyseal plate growth of a longer leg of an LLD patient, thereby solving the leg length discrepancy problem of the patient;

(2) The epiphyseal plate clamping device of the present invention can install a longitudinal tethering effect for epiphyseal plate growth, thereby avoiding tissue damage in the epiphyseal plate from too much compression and avoiding implant loosening from continuous epiphyseal plate growth;

(3) The epiphyseal plate clamping device of the present invention can make use of a medial device coupled with a lateral device to produce symmetrical tethering of epiphyseal plate growth, thereby avoiding bone deformity from asymmetric suppression of the epiphyseal plate; and

(4) The epiphyseal plate clamping device of the present invention can provide a guiding system to accurately install the device with minimum surgical injury.

While the invention has been described by way of example and in terms of preferred embodiments, it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

In summation of the above description, the present invention herein enhances the performance over the conventional structure and further complies with the patent application requirements and is submitted to the Patent and Trademark Office for review and granting of the commensurate patent rights.

What is claimed is:

1. An epiphyseal plate clamping device, which comprises:
a pair of clamping pieces each having at least two through holes; and
at least two screw assemblies to correspond to the at least two through holes of the pair of clamping pieces;
wherein the pair of clamping pieces are used to be placed upon two sides of an epiphyseal plate zone of a longer leg of an LLD patient, and the at least two screw assemblies are used to penetrate through a femur or tibia of the longer leg along at least two paths guided by the at least two through holes of the pair of clamping pieces to clamp an epiphyseal plate, thereby retarding a growth of new bone from the epiphyseal plate.

2. The epiphyseal plate clamping device as disclosed in claim 1, wherein at least one said screw assembly has an internally threaded sleeve and an elongated screw, the elongated screw having a length greater than a width of the longer leg at the epiphyseal plate zone, and when in operation, the elongated screw penetrates through the femur or tibia via a guiding path provided by a guiding pin, which is previously inserted through the femur or tibia and two corresponding through holes of the pair of clamping pieces at opposite sides of the femur or tibia, to be engaged with the internally threaded sleeve.

3. The epiphyseal plate clamping device as disclosed in claim 2, wherein at least one said screw assembly has a guiding sleeve disposed in one of the through holes of one said clamping piece for the elongated screw to pass through.

4. The epiphyseal plate clamping device as disclosed in claim 2, wherein at least one said screw assembly has a pair of screws, and when in operation, the pair of screws are screwed into the femur or tibia through two said through holes of the pair of clamping pieces that are disposed at opposite sides of the femur or tibia.

5. The epiphyseal plate clamping device as disclosed in claim 4, wherein the elongated screw and the pair of screws are made of stainless steel or titanium alloy.

6. The epiphyseal plate clamping device as disclosed in claim 3, wherein the guiding sleeve is permanently or detachably combined with the through hole.

7. The epiphyseal plate clamping device as disclosed in claim 3, wherein the internally threaded sleeve is permanently or detachably combined with the through hole.

8. The epiphyseal plate clamping device as disclosed in claim 1, wherein the pair of clamping pieces is extensible.

9. The epiphyseal plate clamping device as disclosed in claim 8, wherein the pair of clamping pieces each has a stretchable frame body.

10. The epiphyseal plate clamping device as disclosed in claim 9, wherein the stretchable frame body is made of stainless steel or titanium alloy.
